(19) United States
(12) Patent Application Publication
(10) Pub. No.: US 2019/0183406 A1
(43) Pub. Date: Jun. 20, 2019

(54) Prevention and Treatment of Sepsis Using a Simulated Exercise Device

(71) Applicants: Jose Antonio Adams, Miami, FL (US); Marvin A. Sackner, Miami, FL (US)

(72) Inventors: Jose Antonio Adams, Miami, FL (US); Marvin A. Sackner, Miami, FL (US)

(21) Appl. No.: 15/849,494

(22) Filed: Dec. 20, 2017

(51) Int. Cl. A61B 5/00; A63B 22/00; A63B 22/02

(52) U.S. Cl. CPC A61B 5/412; A63B 22/0025; A63B 21/4037; G01N 2800/26; A63B 22/0242

(57) ABSTRACT

A method of treating sepsis and septic shock is using a passive simulated exercise device is disclosed. The exercise device functions to increase pulsatile shear stress (friction) to the vascular endothelium. Increased shear stress stimulates release of beneficial mediators from the endothelium into the circulation that promote integrity of the endothelial barrier as well as to prevent its disruption by suppressing inflammation and oxidative stress. The latter is the basis for the deleterious effects of sepsis and septic shock. Failure to control integrity of the endothelial barrier promotes excessive leakage of fluid and proteins from the vasculature into surrounding interstitial fluid that can rapidly escalate into major morbidity, shock and death.

### BACKGROUND OF THE INVENTION

### 1. Field of the Invention

The present invention is directed to a method of preventing and treating sepsis and septic shock using a motorized machine such as a simulated passive exercise device which increases pulsatile shear stress (friction) to the vascular endothelium of a subject.

### 2. Discussion of Background

Sepsis, produced by infectious diseases, is a leading cause of death in the United States and the most common cause of death among critically ill patients in non-coronary intensive care units (ICU). Respiratory tract infections, particularly pneumonia, are the most common site of infection, and associated with the highest mortality. Recent studies suggest that acute infections worsen preexisting chronic diseases or result in new chronic diseases, leading to poor long-term outcomes in acute illness survivors. Elderly males, African Americans, and individuals with pre-existing chronic health conditions are particularly prone to develop severe sepsis. Therefore, preventative strategies should be strongly considered in such vulnerable populations but owing to the safety and potential of the invention described in this submission, even a healthy population may benefit.

In United States, the estimated incidence of sepsis is from 900,000 to 3,100,00 cases yearly. This was associated with 250,000 to 375,000 deaths per year and included septic shock. Approximately half of these cases occurred outside the ICU. One-fourth of patients who develop sepsis die during their hospitalization. Septic shock is associated with a death rate of 45% in U.S, Australia 22%, Germany 60% and The Netherlands 50%.

Sepsis is life-threatening organ dysfunction caused by a dysregulated host response to infection, Severe Sepsis is included in this definition.

Septic shock is a subset of sepsis in which profound circulatory, cellular and metabolic abnormalities are associated with a greater risk of mortality than with sepsis alone; its clinical criteria is hypotension requiring use of vasopressors to maintain mean arterial pressure>65 mmHg and having a serum lactate>2 mmol/1 persisting despite adequate fluid resuscitation.

Critical Facts about Sepsis
Sepsis is the body's often deadly toxic reaction to infection that affects over 26 million people worldwide each year.
It is the largest killer of children and newborn infants in the world.
Sepsis is a medical emergency that requires urgent attention and treatment.
Mortality increases 8% every hour that treatment is delayed.
Sepsis contributes to 1 in every 2 to 3 deaths in U.S. hospital, and most of these patients are septic on admission.
Sepsis is usually managed by supportive therapies that include antibiotics, fluid replacement, corticosteroids and mechanical ventilation.
Sepsis disrupts the endothelial barrier, creating leakage of fluid and solutes into interstitial spaces, reduces microcirculation, and produces intense inflammatory and oxidative stress responses that challenge targeted therapies.

Human Cost
More than 1.6 million individuals are hospitalized in the U.S. each year—one every 20 seconds and incidence is rising 8% every year.
Sepsis is the #3 leading cause of death in the U.S.
About 1.3 million patients survive but are often left with chronic kidney and liver disease as well as cognitive impairment.
59% (age 50+) with sepsis suffer lasting disabilities.
62% with primary diagnosis are readmitted within 30 days of discharge.
More than 42,000 children develop severe sepsis each year and 4,400 of these children die, more than from cancer.
Maternal mortality—death rates from sepsis increasing in pregnant women.
Adults, including baby boomers (age over 45 years), hospitalization's rate for sepsis increased 180% in 2012.
Seniors (65+) are more likely to be hospitalized with sepsis and are significantly more likely to die from sepsis.

Economic Cost
Sepsis is the #1 cost of hospitalization in the U.S.—about $20 Billion each year.

Awareness
Just 44% of U.S. adults have heard of sepsis.

Treatment Trials

The treatment of sepsis has been called “The graveyard of the pharmaceutical industry.” Since the 1960s, over 100 targeted clinical trials of drugs have failed to significantly alter sepsis mortality.

Early goal-directed therapy of sepsis received initial enthusiasm but following its adaption in many tertiary care facilities, did not alter its course and has not achieved mainstream acceptance. Its goals were mostly hemodynamic that required invasive, labor-intensive, monitoring interactions that often could not be justified in clinical practice. For example, one goal was to achieve a mean arterial blood pressure (MAP) greater than 65 mmHg with pharmacologic pressers or fluid therapies. However, lower blood pressures might have been just as effective because septic shock is associated with abnormally low microvascular perfusion.

The search for a pharmacological remedy for sepsis has mainly relied on survival from death in mice or rats before or after administration of provocative doses of lipopolysaccharide (LPS), the outer wall of E. coli bacteria. Potential treatments counteract the inflammatory cytokines elevated by sepsis which disrupt the endothelial barrier, produce intense inflammation, increase oxidative stress and shortens life span in a dose-dependent way. Our review of survival data in the scientific literature revealed that only 0 to 20% of mice survive 48 hours with LPS intraperitoneal doses of 30 to 40 mg/kg without treatment. Anti-inflammatory, chemotherapeutic, or pesticide agents administered after LPS provide 80 to 90% survive up to 48 hours. However, none of these agents are approved for human use and their use in human sepsis would require complex, human toxicologic studies. Further, effectiveness of such treatments prior to LPS administration for sepsis prevention has not been reported. Clearly, a safe intervention that can be brought swiftly to a clinical trial with a track record in mice given a lethal dose of LPS is sorely needed.

### SUMMARY OF THE INVENTION

A method of treating a patient for septic shock or preventing sepsis in the patient by using a motorized machine, said machine having a housing, an axis-defining mechanism coupled to the housing, the axis-defining mechanism configured to define a rocking axis, at least one pedal positioned to receive a foot of the user and mounted on the rocking axis for rocking movement of the at least one pedal; a motor arranged within the housing, the motor configured to generate rotational motion to an output shaft of the motor, a pedal rocking mechanism coupled to the output shaft and driven by the motor, the pedal rocking mechanism being configured to translate the rotational motion generated by the motor to reciprocating rocking motion of the at least one pedal about the rocking axis, and at least one bumper, height-adjustably coupled to the housing, located spaced apart from a bottom portion of the at least one pedal, the reciprocating rocking motion of the at least one pedal provided by the pedal rocking mechanism providing a positive application of force for moving the bottom portion of the pedal towards and away from the at least one bumper, wherein the motor, the pedal rocking mechanism, the at least one pedal and the at least one bumper are configured so as to cooperate to apply, by the pedal rocking mechanism, the positive application of force to the at least one pedal so as to, during operation of the motor, cause the bottom portion of the at least one pedal to tap against the at least one bumper so as to provide pulsatile acceleration to the bottom of the user's foot, said pulsatile acceleration having a force sufficient to increase pulsatile shear stress to the endothelium, of sufficient magnitude to cause the release of beneficial mediators; the method comprising placing the patient in one of a seated and supine posture with the patient's foot on the at least one pedal, securing the patient's foot to the at least one pedal, and applying power to the device to cause the at least one pedal to move, at a therapeutically-effective speed, for a therapeutically-effective amount of time, towards and away from said bumper to cause the pedal to contact said bumper.

In one embodiment, in the case of sepsis prevention, the method is performed twice daily for the therapeutically-effective amount of time.

In another embodiment, in the case of potential sepsis exposure, the method is performed twice daily, each time for the therapeutically-effective amount of time, the therapeutically-effective amount of time being at least 30 minutes, and wherein the twice daily performance of the method beginning at least three days prior to the potential exposure to sepsis.

In yet another embodiment, in the case of sepsis treatment, the method is performed upon detection of one of an unexpected fever, elevated heart rate and respiratory rate of the patient.

The various features of novelty which characterize the invention are pointed out with particularity in the claims annexed to and forming a part of the disclosure. For a better understanding of the invention, its operating advantages, and specific objects attained by its use, reference should be had to the drawing and descriptive matter in which there are illustrated and described preferred embodiments of the invention.

### BRIEF DESCRIPTION OF THE DRAWINGS

In the drawings:

FIGS. 1A and 1B show an experimental motion platform for use on mice to increase pulsatile shear stress to the endothelium;

FIG. 2 is a plan view of an apparatus in accordance with an embodiment of the present invention;

FIG. 3 is a section view taken along the lines 3-3′ in FIG. 2;

FIG. 4 is a section view taken along the lines 4-4′ in FIG. 2;

FIG. 5 is a section view taken along the lines 5-5′ in FIG. 2

FIG. 6 is a section view taken along the lines 6-6′ in FIG. 2;

FIG. 7 is a perspective view of the apparatus of FIG. 1 with the top cover and one foot pedal removed;

FIG. 8 is a perspective view of the underside of a foot pedal according to one embodiment of the present invention;

FIGS. 9 and 10 are graphs showing that the CONT & pGz did not significantly differ for the first 360 minutes after LPS but diverged at 420 minutes with signs of illness until all CONT mice were dead at 24 hours.

FIG. 11 is a composite graph for the preconditioning experiments to show the role that NO₂ plays in sepsis.

FIGS. 12 and 13 are graphs depicting in mice heart muscle Western Blot values in Optical Units (O.U.) referenced to protein/GADPH/SHAM at 90 and 360 minutes, respectively, after initiation of investigation.

FIGS. 14 and 15 are graphs depicting in mice heart muscle Western Blot values in Optical Units (O.U.) referenced to protein/GADPH/SHAM at 90 and 360 minutes, respectively, after initiation of investigation.

FIGS. 16 and 17 are graphs depicting the effects of pGz Pre- and Postconditioning on TIE2 Expression/Phosphorylation and Microvascular Permeability at 90 and 360 minutes, respectively.

FIG. 18 is a graph depicting results of in vivo injection of Evans Blue dye in mice to estimate fluid leakage from destruction of the endothelial barrier through post-mortem analysis.

### DETAILED DESCRIPTION OF THE PRESENTLY PREFERRED EMBODIMENTS

Reference is made herein to the passive simulated exercise apparatus/device disclosed in U.S. Pat. No. 9,622,933, the entire content of which is incorporated by reference herein.

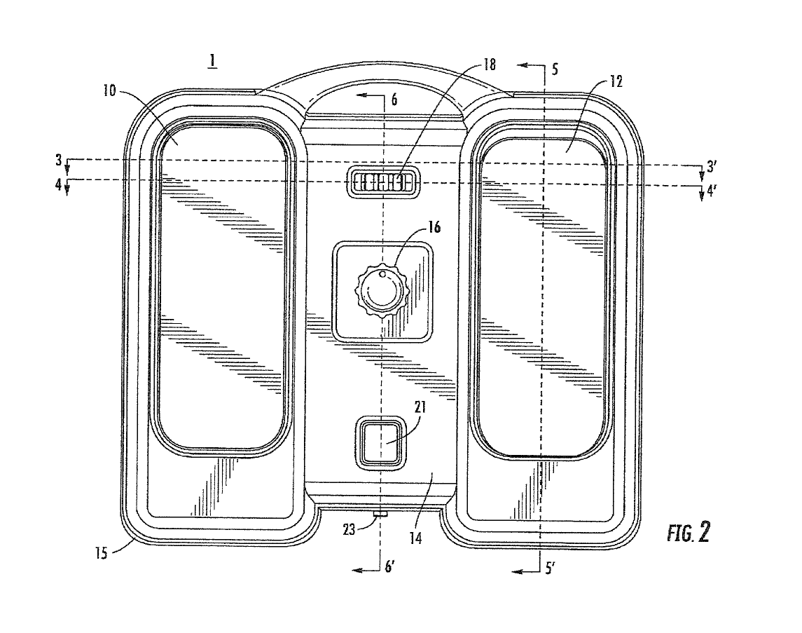

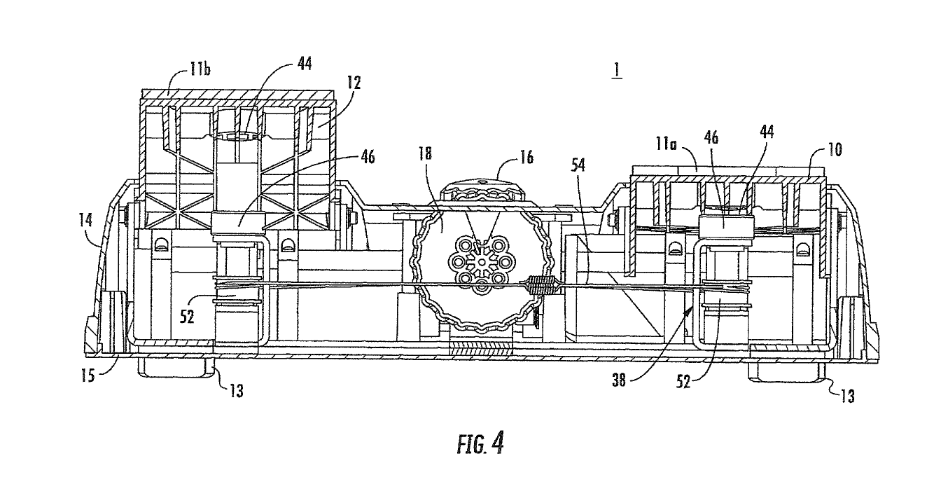

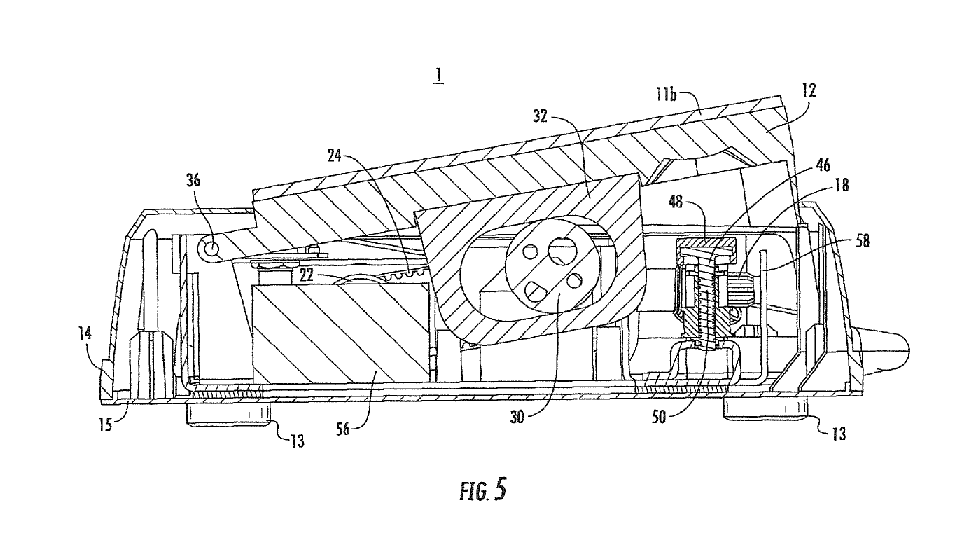

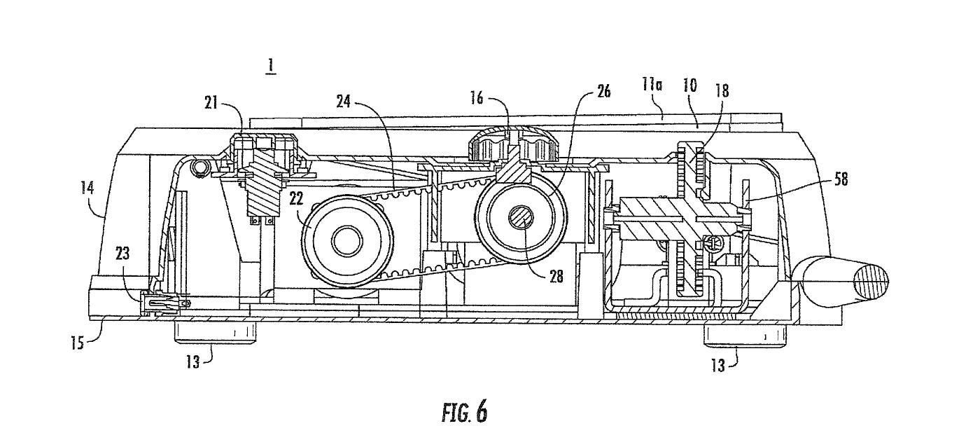

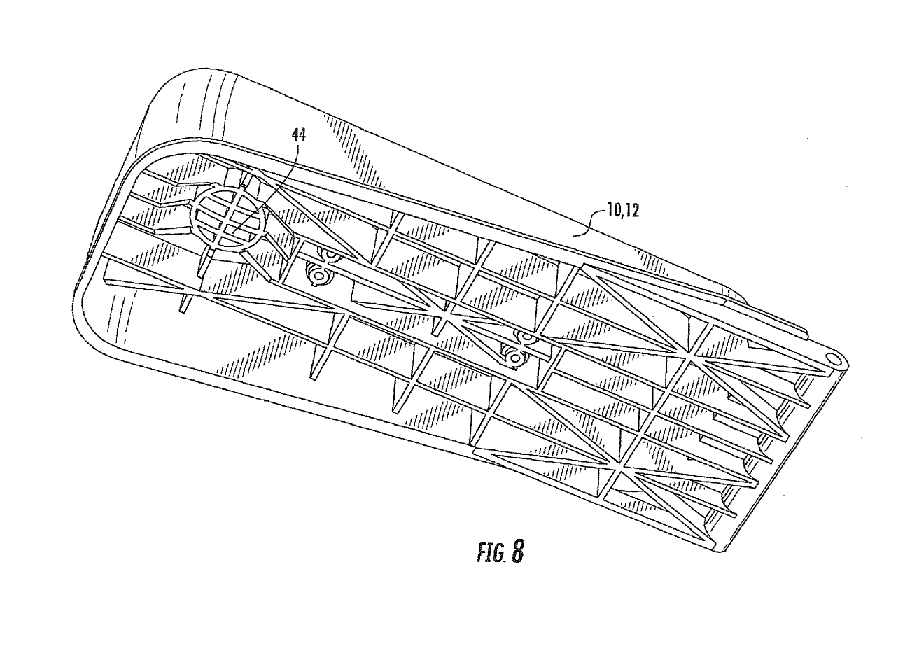

FIGS. 2-8 and 11 show an exemplary embodiment of passive simulated exercise apparatus for use in treating sepsis and septic shock. The apparatus includes a pair of foot pedals, each of which are driven to up and down, rocking movement about an axis transverse to the feet, preferably alternating, i.e., anti-phase, motion of the two foot pedals. The apparatus is configured such that each movement of the foot pedals can be associated with a percussive contact of a portion of the underside of the foot pedal, which percussive contact passes along to the user a pulsatile impact which, as is discussed above, increases shear stress to mechanically stimulate the endothelial cells to increase the activity of genes responsible for release of beneficial mediators. In particular, the tapping simulates the beneficial effects that occur, for example, while running, in which Pulsatile shear stress (PSS) is increased by addition of pulses generated by the tapping. By virtue of this feature of the present invention, a pulse is added to the circulation that is superimposed upon the body's own pulses and is detected in the radial arterial pressure waveform.

In a typical operation of the apparatus, the feet will be placed on the pedals such that the toes will be raised (and then lowered) in relation to the heels by the rocking of the pedals, and the tapping applied to the toe portion of each foot. However, the apparatus is advantageously symmetrical in design so as to permit the heels, rather than the toes, to be raised and lowered, by the user turning the apparatus around 180.degree. and placing his or her feet in the opposite direction. Such reversed usage of the apparatus results in the pulse being delivered to the heel of the user rather than to the toe.

As can be seen in FIGS. 2-8, the apparatus 1, in accordance with an embodiment of the present invention, includes a housing top 14, a housing bottom 15, and left and right foot pedals, 10 and 12, having surfaces 11a and 11b, respectively, for receiving the feet of a user. The bottom of the apparatus preferably includes bottom stabilizer posts 13, e.g., made of rubber, to contact the ground, provide a leveling function and prevent slippage of the apparatus during use.

As can be seen, for example, in FIG. 2, the apparatus 1 may include a speed adjustment control 16, which can vary the speed of the up and down motion of the pedal 10 and 12. The adjustment control can be in the form of a knob, switch, lever or other user-selectable device. As an example, the control 16 is depicted in the figures as a knob. The housing top 14 and housing bottom 15 are preferably coupled to one another using screws 17.

A force adjustment control 18 is provided, a portion of which is accessible through an opening in the housing top 14 to allow adjustment of the intensity of tapping or striking force provided by the motorized machine or device 1. The ability to adjust the speed of the up and down motion of the pedals 10, 12 is optional and may be omitted.

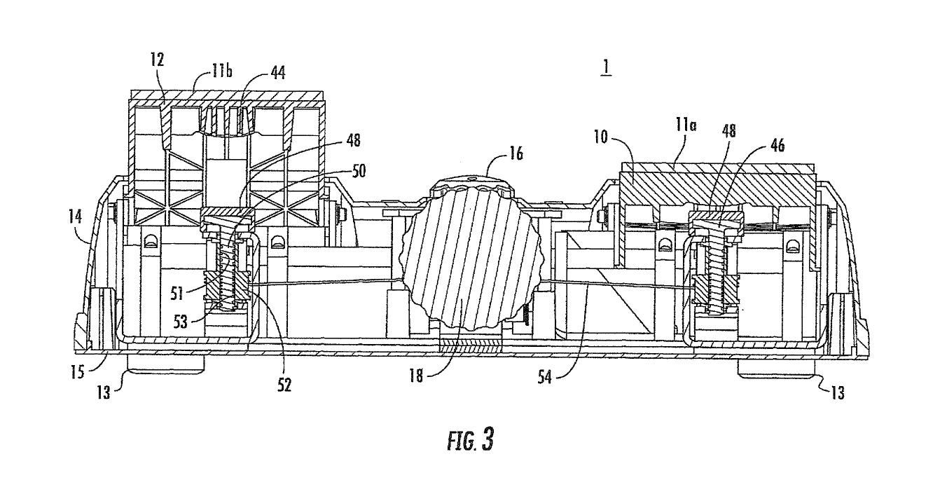

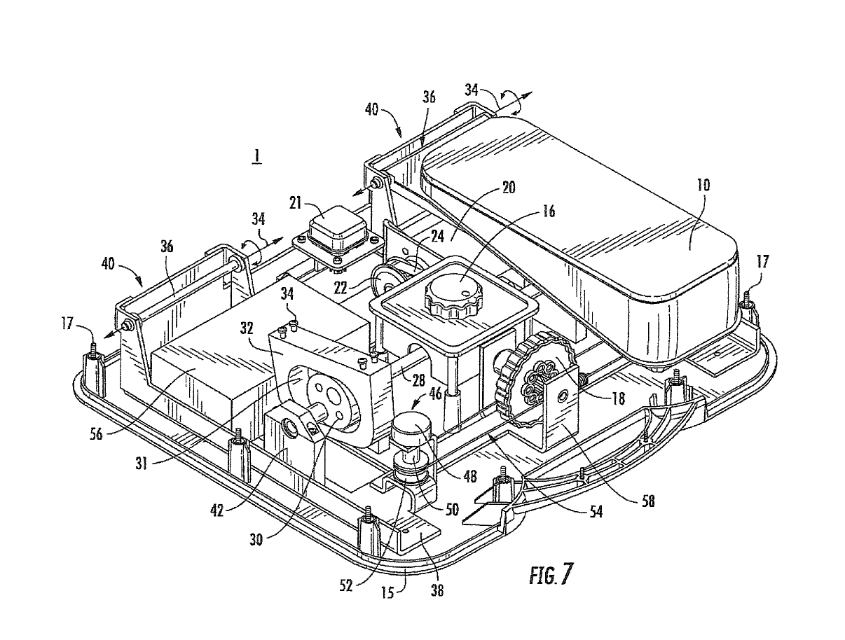

The interior workings of the motorized machine, exercise device 1 can be seen in the sectional views of FIGS. 3-6, as well as the perspective view of FIG. 7, which shows the interior without the housing top 14 and without right pedal 12. As shown in these figures, the interior of the device 1 includes mechanical and electrical elements that cooperate to cause the pedals to rockingly reciprocate, e.g., anti-phase to one another, between up and down positions, the pedals being rotatable, preferably at a rearward portion of each pedal, about a common axis.

The rocking motion for the movement of the pedals is provided in the first embodiment by a driving mechanism that includes a motor 20, the drive shaft of which drives a motor pulley 22. A stop/start button 21 is preferably provided to start the operation of the motor. The motor 20 is preferably a motor of a well-known type, such as a DC brushless motor, of a power sufficient to drive pedals of the apparatus. Power to the motor 20 is supplied, e.g., using power connector 23, or by disposable or rechargeable batteries, not shown.

The motor pulley 22 contacts a belt 24 which is also contacting a camshaft pulley 26. The belt transfers rotational motion of the motor pulley 22 to provide rotational motion to the camshaft pulley 26.

This rotation in turn causes a camshaft 28, arranged along an axis perpendicular to the camshaft pulley 26 and transverse to the feet, to rotate. A cam 30 is eccentrically coupled to each end of the camshaft 28. The eccentricity is provided, in the present embodiment, by the camshaft 28 coupling with the cam 30 in an off-center manner, that is, coupling to the cam 30 at a point on the cam 30 axially offset from the center of the cam 30. The off-center coupling causes eccentric rotating motion of each cam 30. While the cam 30 and the camshaft 28 are shown in the first embodiment as being distinct elements, the cam 30 can also be an integrally formed portion of each end of the camshaft 28

To translate the rotational motion of the camshaft 28 to the up and down motion of the pedals, each cam 30 is arranged in a channel 31 provided in a pedal coupling member 32. The channel 31 is configured such that the eccentric motion of the cam 30 causes the coupling member 32 to reciprocate, such that a front end of the coupling member 32 moves up and down to a greater extent than the rear end of the coupling member 32.

The top of each coupling member 32 is affixed, for example, by screws 34, to the underside of the respective foot pedals 10 and 12. The cams 30 are arranged in the channel 31 of the respective pedal coupling members 32 such that the motion provided to the two pedal coupling members 32 by virtue of the eccentricity of the cams 30 at each end of the camshaft 28, generates alternating, i.e., anti-phase, reciprocating up and down motion of the pedals 10 and 12, so that, preferably, when one pedal is moving up, the other is moving down. However, in a variation of this configuration, the cams can be configured to provide in-phase movement of the pedals.

In the above-described manner, the motion of the camshaft 28, driven by the pulleys 22 and 26 and the motor 20, drives the pedals in an up and down motion about a common axis 34. The common axis 34 is preferably provided towards the rear of each pedal 10, 12 being rotatably mounted around a pedal axle 36, disposed along the common axis 34. While the disclosed embodiment shows the common axis disposed at an extreme end of each pedal, the invention is not limited to this configuration, and the device could be alternatively set up with the axis of rotation located away from an extreme end, while still providing the rocking motion.

The motor 20 is mounted on a mounting plate 38, to which various elements of the driving mechanism described above are also coupled, either directly or indirectly. The mounting plate 38 is located between the housing top 14 and the housing bottom 15 and acts as a chassis for mounting internal components of the exercise device 1.

The mounting plate 38 is preferably made of a lightweight metal, for example aluminum, steel, or the like. However any sufficiently strong and lightweight material can used, such as carbon reinforced plastic, or other similar material, that will result in a lightweight travel-friendly device. The mounting plate 38 includes two pedal mounting flanges 40 structured to secure each pedal axle 36 and the rear of each pedal 10, 12. Also coupled to the mounting plate 38 are bearing blocks 42, each of which receives and secures an end of the camshaft 28, or a tubular extension thereof, to allow rotation of the camshaft 28.

As an alternative to the pulley and belt system, the output shaft of the motor 20 can be arranged perpendicular to the camshaft, and a bevel gear configuration used to drive the camshaft. Another variation would use a motor having output shafts along the rotational axis of the camshaft so as to directly drive the camshaft.

Optionally, the motor 20 can be adjustable to increase or decrease the speed of the movement of the pedals. In the speed-adjustable embodiment, a motor controller 56 is provided, which controls the speed of the motor 20 in accordance with the position of the speed adjustment knob 16. Such adjustment is well-known in the art and can be done in any conventional manner, for example by use of a potentiometer controlled by the knob 16, in which the motor speed is varied proportionally to a position of the knob 16, or electrical or digital equivalents thereof. In such configuration, the controller 56 is digitally or otherwise configured to receive information from the knob 16 and, based on this information, control the speed of the motor 20.

To provide beneficial tapping pulses to the user, each pedal 10, 12 is configured to contact a top portion of a bumper 46, at an inside contact surface 44 of each pedal, at the bottom of the downward toe stroke of each pedal provided by the reciprocating motion of the coupling members 32. Each bumper 46, one arranged under each pedal respectively, includes a bumper cover 48, for example made of rubber, and a bumper body 50, the lower part of which is a threaded cylindrical portion having threads 51.

The bumper body 50 is threadingly coupled to the mounting plate 38 such that rotation of the bumper body 50 effects an adjustment of its height with respect to the bumper body 50, as well as its proximity with respect to the contact surface 44 of the pedal 10, 12. In particular, to achieve adjustment of the height of the bumper 46, an annular screw jack 52 is configured such that inner threads 53 of each annular screw jack 52 mate with corresponding threads 51 of the cylindrical portion of the bumper body 50, so as to cause, upon a rotation of the annular screw jacks 52, a corresponding rotation of the bumper body 50, causing a change in the height of the bumper body relative to the mounting plate 38.

Each screw jack 52 having threads 53 is coupled to a tension cable 54 that wraps around the screw jack 52. The tension cable 54 is adjusted by the force adjustment control 18. The force adjustment control can be in the form of a knob, switch, lever or other user-selectable device. As an example, the control 18 is depicted in the figures as a knob. The force adjustment control knob 18 is coupled to the tension cable 54 so that adjustment of the knob 18 in a first direction bumpers 46, by twisting the screw jack 52 in one direction, e.g., clockwise, and adjustment of the control knob 18 in a second direction lowers bumpers 46, by twisting the screw jack 52 in an opposite direction, e.g., counter-clockwise. The knob 18 is preferably coupled to the mounting plate 38 at a dedicated rectangular portion 58 of the mounting plate 38, as can be seen in the figures.

The configuration of the bumper 46 and the control knob 18 allows for adjustment of the intensity of striking of the pedal 10, 12, in particular the contact surface 44, with the top of the bumper 46 by the turning of the control knob 18. The higher the position of the top of the bumpers 46, results in an increase of the pulsatile force applied to the bumpers 46. In a preferred embodiment the height of the bumper 46 is adjusted to allow for tapping that provides a range of pulsatile acceleration having a force sufficient to increase pulsatile shear stress to the endothelium, of sufficient magnitude to cause the release of beneficial mediators, such as nitric oxide, prostacyclin, tissue plasminogen activator, adrenomedullin, SIRT1, Brain and Glial Derived Neurotrophic Factors (BDNF & GDNF), Kruppel Like Factor 2, Superoxide Dismutase, Glutathione Peroxidase 1, Catalase, Total Antioxidant Capacity, Anti Apoptotic Proteins: p-Akt, Bcl2, and Bcl2/Bax, HSP27. Preferably, such effects can be provided with an acceleration of about 0.1 g to 0.5 g.

Such tapping to the feet provided by the apparatus can increase pulsatile shear stress as related to the addition of pulses into the vascular circulation, heart, lymphatic channels, interstitial spaces, skeletal muscle and bone interstices, as well as slight increases of cyclic strain to the blood vessels and lymphatic channels.

The tapping is also settable so as to increase the activity and content of endothelial nitric oxide synthase (eNOS) in blood vessels, heart and skeletal muscle, as well as to increase the activity of neuronal nitric oxide synthase (nNOS) in the heart and skeletal muscle. Moreover, using the apparatus repeatedly adds pulses and minimally increases cyclic strain, by the striking of the flat, padded, hard surface of the bumper 46 with the foot pedals, to the body's fluid filled channels over the body's own pulse such that even during periods when pulses are not imparted, bioavailability of the beneficial mediators is greater than the preoperational period.

Moreover, adding the pulses, using the striking of the bumper with the foot pedals, to the body's fluid filled channels over the body's own pulse stimulates endothelial release of at least one of nitric oxide, prostacyclin, tissue plasminogen activator (t-PA), adrenomedullin, endothelial dependent hyperpolarizing factor (EDHF), endothelial dependent relaxing factor, endothelial growth factors, and transcription factors, etc.

The device disclosed in U.S. Pat. No. 9,622,933 can increase pulsatile shear stress (friction) to the vascular endothelium. This non-invasive device passively increases pulsatile shear stress to the endothelium of individuals seated or lying down or those at risk or suffering from acute illnesses that have as a major component endothelial barrier dysfunction. Increased pulsatile shear stress mechanically distorts endothelial cells to activate genes that increase release of nitric oxide, prostacyclin, SITR1, and adrenomedullin among others into the circulation as well as to up-regulate expression of Tie-2 at endothelial cell junctions. These mediators and factors stabilize the endothelial barrier to aid in reducing morbidity and mortality associated with endothelial barrier dysfunction as well as to reduce oxidative stress and inflammation. Sepsis universally causes endothelial cells to release deleterious, tissue damaging mediators and molecules into the circulation such as endothelin-1, and free radicals such as superoxide (O2-), hydrogen peroxide (H2O2), and peroxynitrite (OONO—) as well as reactive nitrogen species. By increasing shear stress to the endothelium, the device disclosed in U.S. Pat. No. 9,622,933 inhibits release of such harmful elements.

Reversal of endothelial dysfunction using the device disclosed in this patent can be monitored by several technologies, but the acute care setting placed limitations on applications during sepsis. The SphygmoCor system (AtCor Medical, Sydney Australia) in conjunction with a computerized blood pressure system that displays an analog representation of a waveform is one such example. Pulse wave analysis (PWA) uses a validated transfer function to calculate the central aortic waveform from a peripheral, noninvasively-obtained, arterial pressure waveform. The transfer function is a mathematical model that describes the relation between the central and peripheral arterial waveforms. Augmentation is defined as the difference between the second systolic peak (caused by wave reflection) and the first systolic peak (caused by left ventricular contraction). The augmentation index (AIx) is this difference expressed as a percentage of the central pulse pressure. Increasing values of this index indicate a good prognosis as endothelial function improves while decreasing values that approach 0.5% or less a poor prognosis. The SphygmoCor or similar systems that provide aortic waveform analysis aid in decisions for frequency of use of the device disclosed in this patent application.

Endothelial Barrier

The vascular endothelium covers the inner lining of all lymphatic and blood vessels and the heart, thereby acting as a highly widespread “organ” along the 100,000 km of everyone's vascular tree. It is engaged in the regulation of blood pressure, hemostasis, inflammation and defense and in organ-specific perfusion. The endothelium functions as a selective barrier that tightly balances the exchange of water, solutes, macromolecules and mobile cells between the flowing blood and the underlying tissues. This balance is essential to achieve systemic and site-specific vascular homeostasis by responding to a wide variety of physiological and pathological stimuli, resulting in changes of intercellular junctions or trans-cellular transport processes. Apart from their biological relevance in maintaining and regulating barrier function, endothelial cell junctions are major targets in many vascular and infectious diseases and in cell regeneration and repair, as they affect shape changes, barrier breakdown/apoptosis or cell migration, and proliferation.

Alterations in endothelial barrier function play important roles in pathogenesis of many disease states, including inflammation, wound healing, edema, acute lung injury, stroke and cancer. Endothelial barrier maintenance and the response of the quiescent barrier to locally produced vasoactive agents, such as histamine, prostaglandins, thrombin and VEGF is primarily mediated by dynamic changes in cell to cell junctions.

Proof of Concept

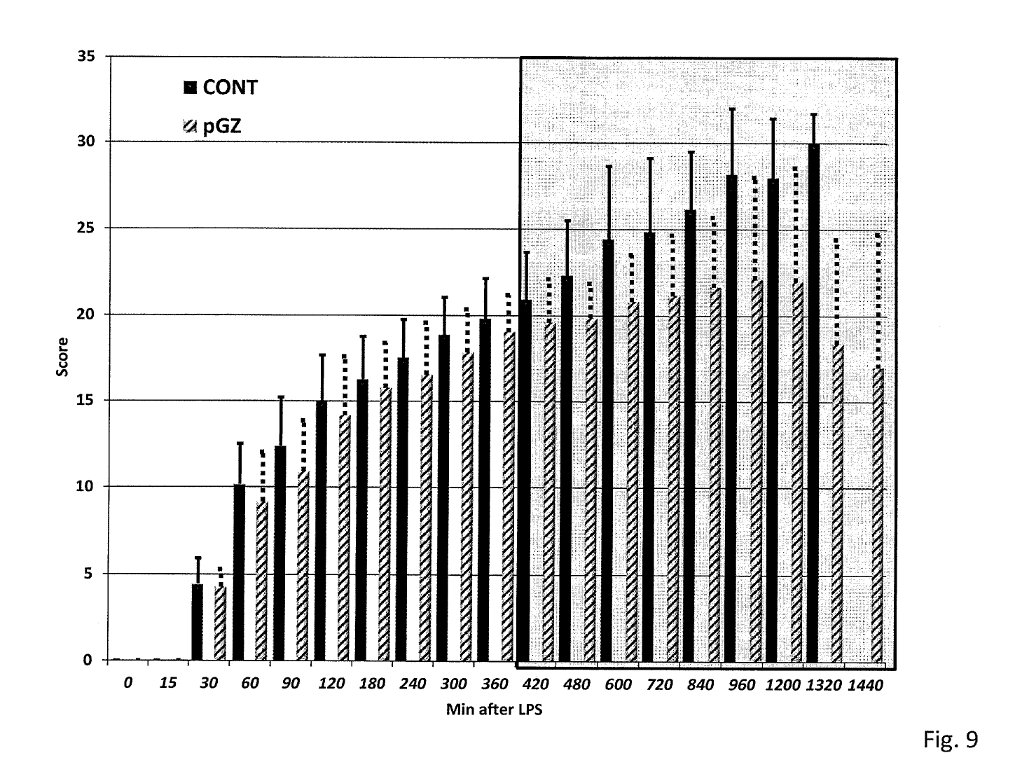

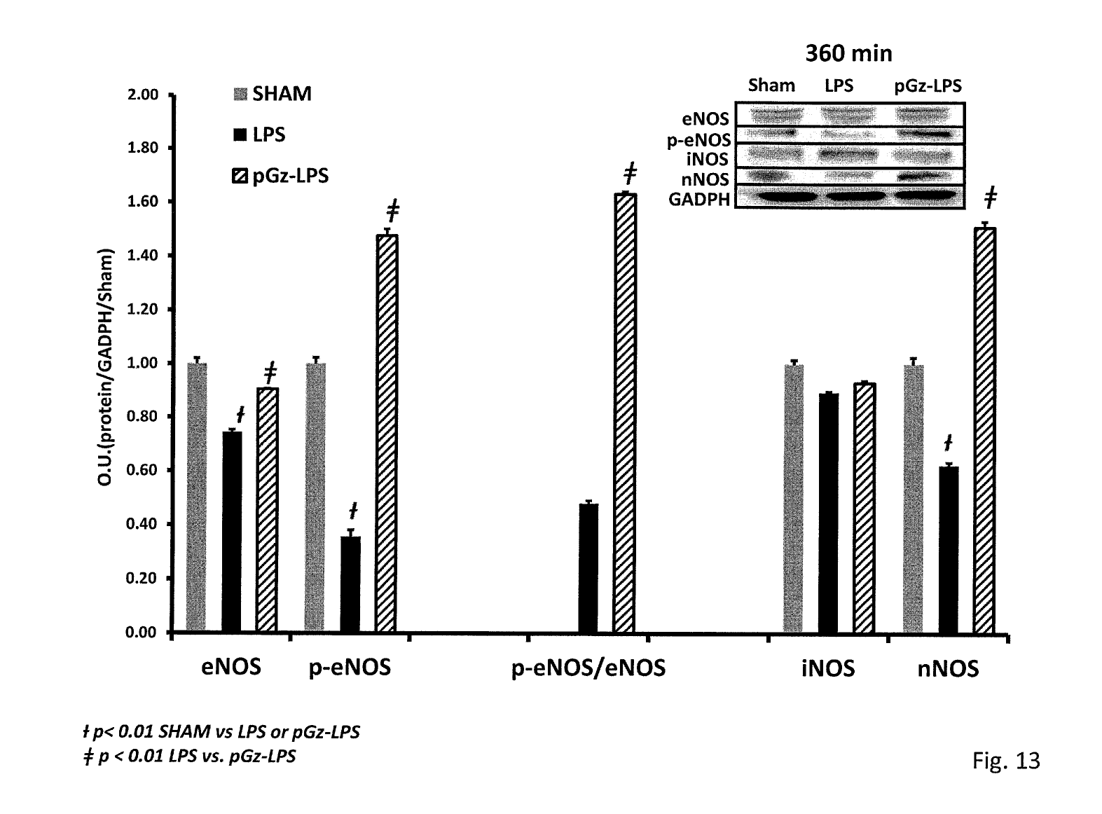

Described herein is a simple, cost-effective means to preserve and maintain endothelial barrier integrity through the increase of substances upregulated and released during operation of a passive simulated exercise device of the type disclosed in U.S. Pat. No. 9,622,933. The administration of a lethal dose of lipopolysaccharide (LPS) to mice and survival from death is the basis for “proof of concept”, as explained below and illustrated in the graphs of FIGS. 9-18.

For this investigation in mice, a motion platform assembly was employed to increase pulsatile shear stress to the endothelium, as disclosed in U.S. Pat. No. 7,090,648, the entire contents of which are incorporated by reference. That patent discloses methods of medical treatment and diagnosis using mediators released by endothelial cells stimulated by external addition of pulses to the circulation. The external pulses produce circumferential shear stress in body fluid channels that subsequently stimulates the endothelial cells to produce mediators that become available for therapeutic and diagnostic purposes. The preferred means of adding external pulses is the mechanical inducement of periodic acceleration of the body or parts of the body by a reciprocating motion platform.

Treatment with a non-invasive, reciprocating motion platform that increases pulsatile shear stress to endothelium (pGz or WBPA) for hours or days prior to or after a lethal dose of polysaccharide (LPS) in mice reduces mortality. [pGz denotes repetitive bouts of periodic acceleration in the Z axis, e.g., head to foot plane whereas WBPA signifies whole body periodic acceleration, a term interchangeable with pGz].

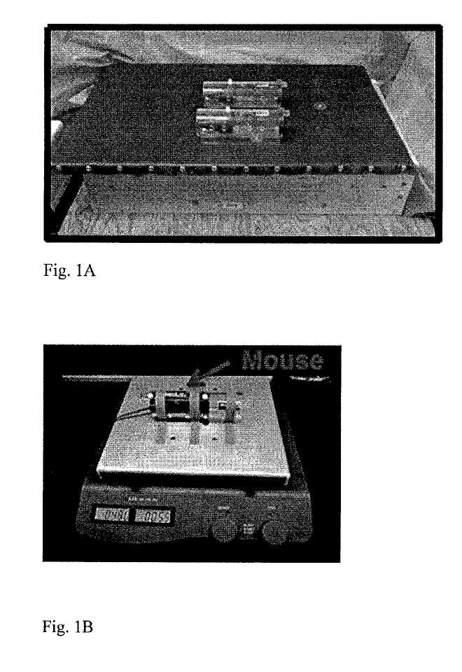

The platform and restraining cylinder are depicted in FIGS. 1A and 1B.

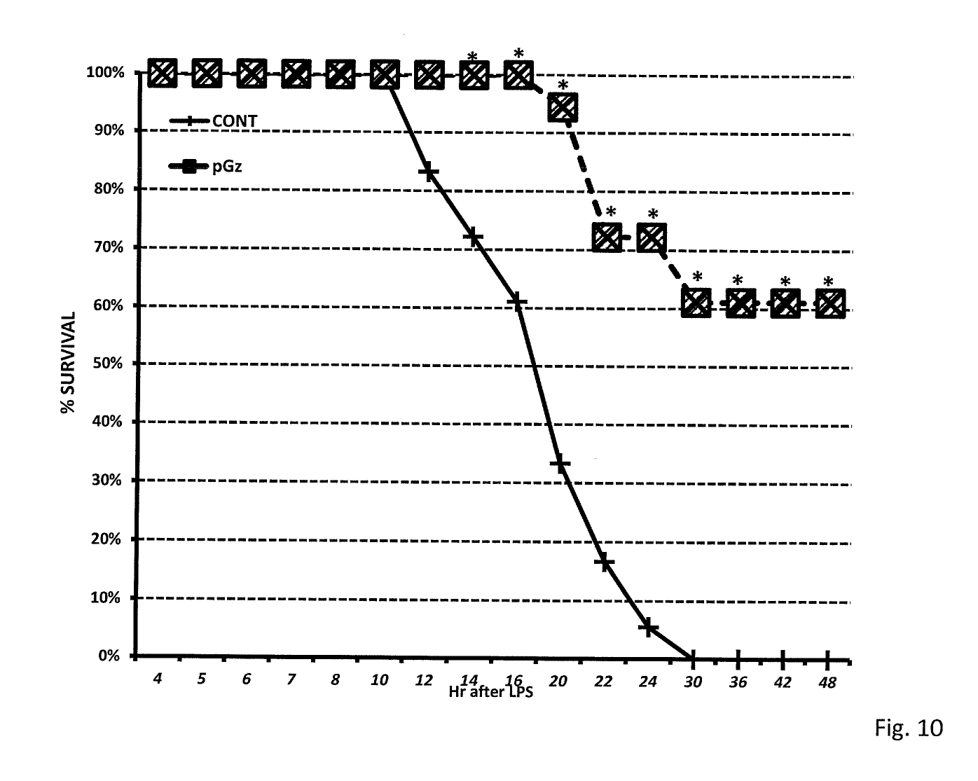

Preconditioning
Wild type mice were used for this investigation.
   18 mice were selected as controls (CONT) and received the same diet and granted unrestricted physical activity as mice used for pGz treatment.
   18 mice were placed on the motion platform, each within a Plexiglas restraining cylinder (figure above) that moved at 480 cycles per second for one hour daily for 3 days (pGz).
Following the final pGz treatment, all mice received intraperitoneal (i.p.) injections of 40 mg/kg LPS and were observed with video monitoring for 48 hours.
A behavioral scoring system was scored as a measure of health in the mice every 30 minutes with 0=normal and 32=death.
As shown in FIG. 9, scores for the CONT & pGz did not significantly differ for the first 360 minutes after LPS but diverged at 420 minutes with signs of illness until all CONT mice were dead at 24 hours.
Also shown in FIG. 9, reduced survival began in untreated mice (CONT) 14 hours after LPS and continued to decline until all CONT mice were dead 30 hours after LPS.
As shown in the composite FIG. 10, mice pre-treated with pGz began to die 20 hours after LPS, but 60% survived for 48 hours, the predetermined outcome duration of the trial.

Role of Nitric Oxide in Sepsis
L-NAME, a nitric oxide synthase inhibitor was orally administered in drinking water in 8 non-LPS administered mice for 7 days and in 8 mice prior to LPS 40 mg/kg i.p. (L-NAME-pGz) and mortality compared to 8 mice given LPS only. As depicted below, L-NAME that blocks nitric oxide release even if combined with pGz produces the same fatal outcome in 14 hours.

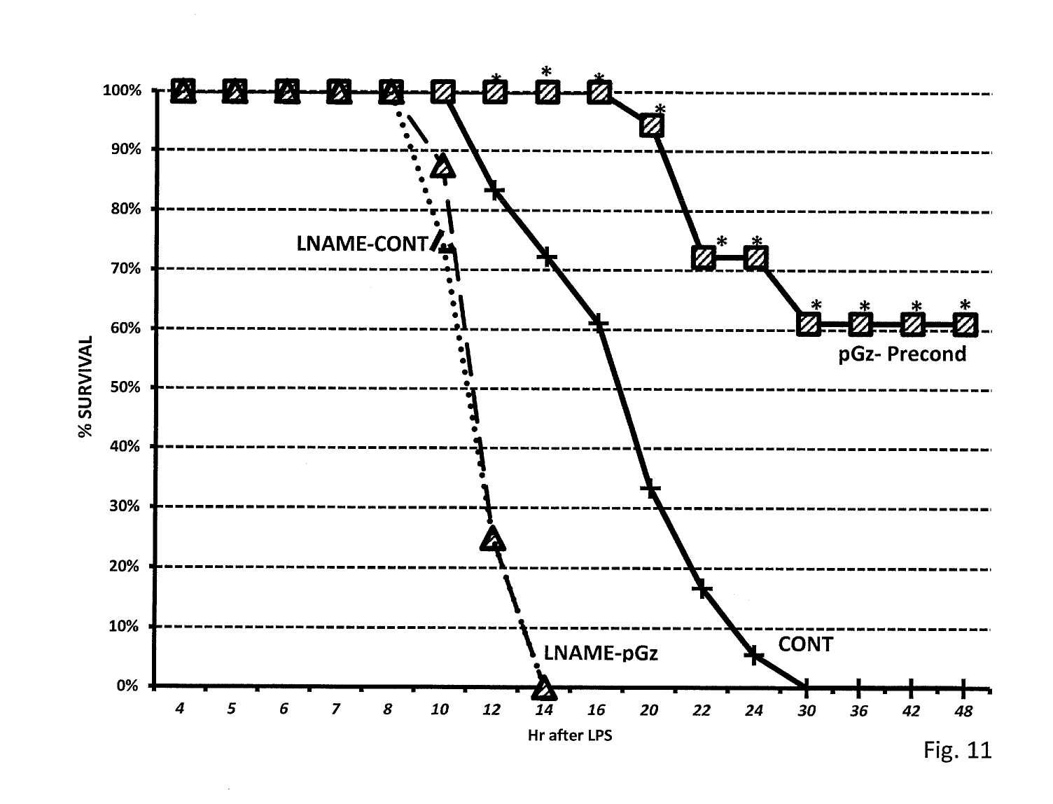

A composite graph for the preconditioning experiments is depicted in FIG. 11.

Nitric Oxide Synthases, Inflammatory and Anti-Inflammatory Factors

a. Material
Three groups of 8 mice were selected for this investigation.
SHAM mice: placed within restraining cylinder.
   4 mice sacrificed at 90 minutes.
   4 mice sacrificed at 360 minutes.
LPS mice: given 40 mg/kg LPS i.p. & placed within restraining cylinder.
   4 mice sacrificed at 90 minutes.
   4 mice sacrificed at 360 minutes.
pGz-LPS mice: pGz for 1 hour within restraining cylinder followed by 40 mg/kg LPS i.p.
   4 mice sacrificed at 90 minutes.

4 mice sacrificed at 360 minutes.

Tissue Analyses

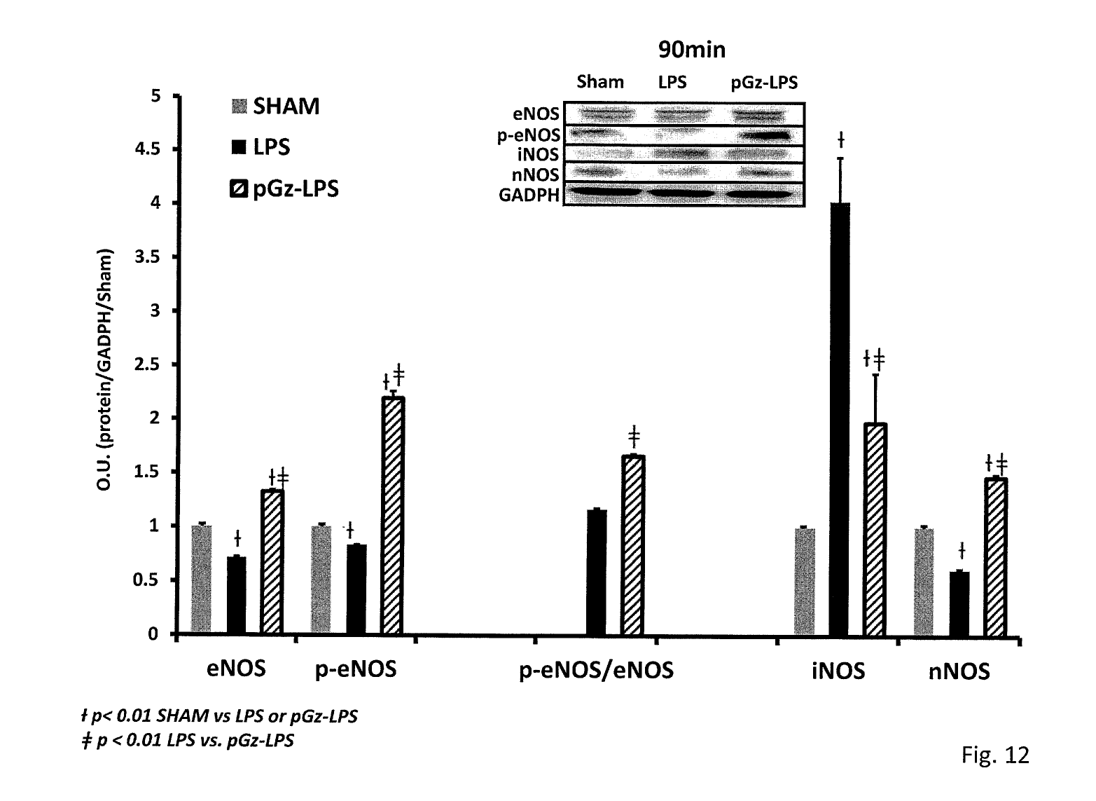

FIGS. 11 and 12 depict in mice heart muscle Western Blot values in Optical Units (O.U.) referenced to protein/GADPH/SHAM at 90 and 360 minutes after initiation of investigation.

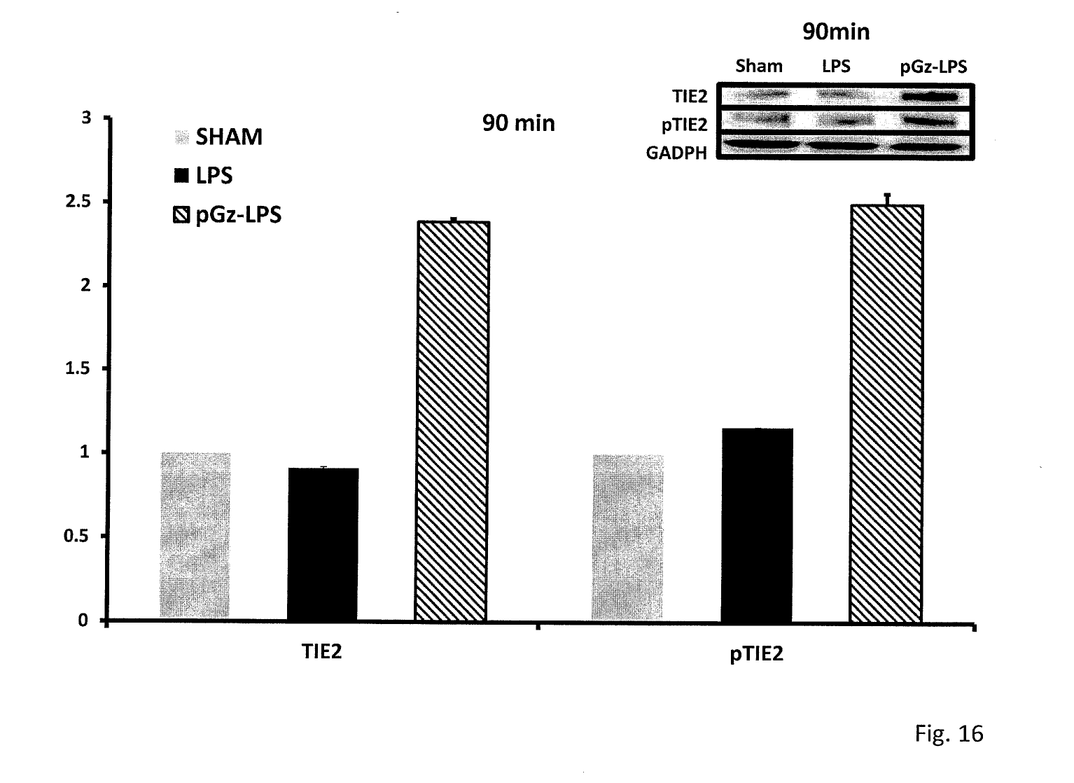

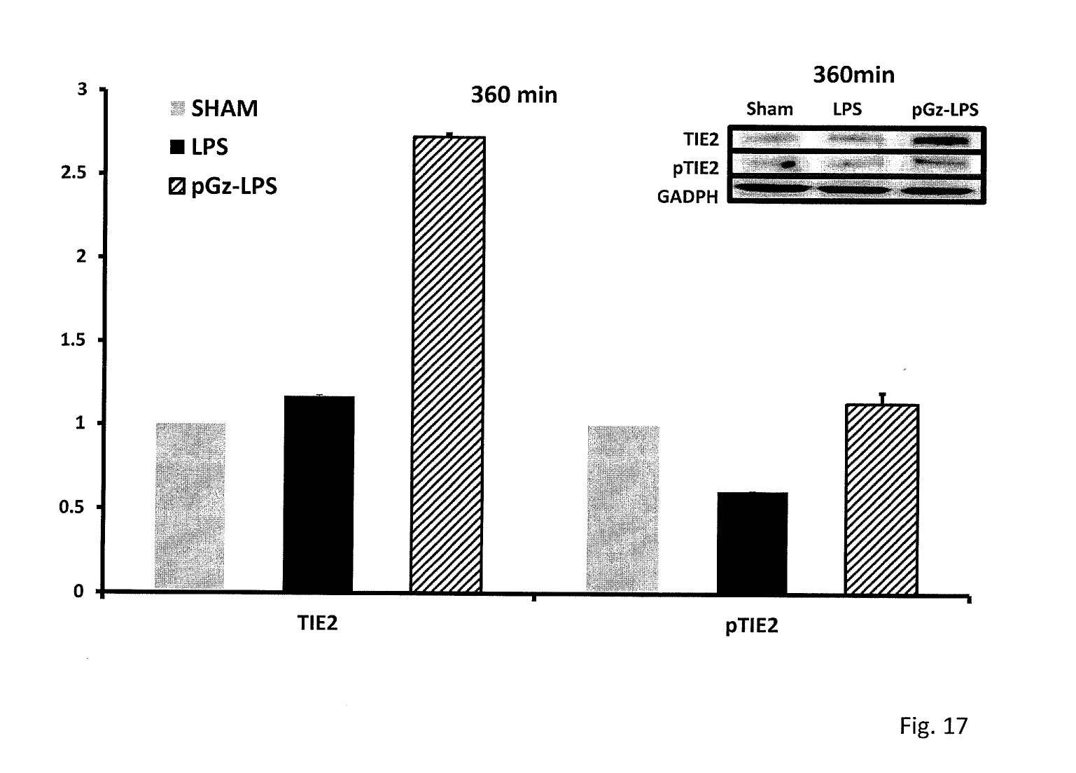

Interpretation of NOS Changes by LPS and pGz
Note that the Y axis scale of FIG. 16 is 40% of the FIG. 17 graph.
pGz was administered at the beginning and 6 hours later of the LPS challenge.
Phosphorylated eNOS (p-eNOS), the active, beneficial form of eNOS, in pGz-LPS is lower at 360 than 90 minutes.
iNOS, the inflammatory form of NOS, in pGz-LPS is approximately the same at 90 and 360 minutes.
nNOS is the content of a possible beneficial form of NOS in cardiac muscle and is approximately the same level at 90 and 360 minutes.
Conclusion: To increase p-eNOS that suppresses inflammatory factors, either longer duration, intermittent or more delayed pGz treatments should be employed; such a schedule can be facilitated with the app communicating with the Passive Simulated Jogging Device,

Cytokines and Inflammatory Factors:

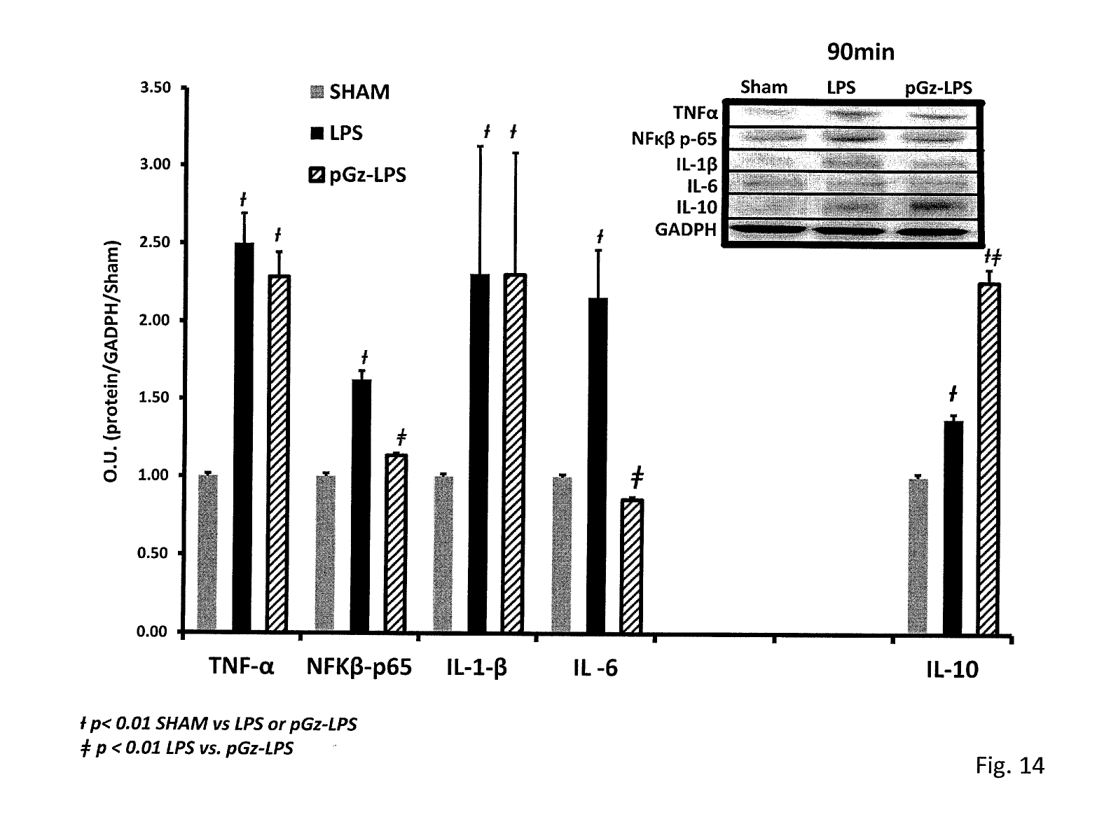

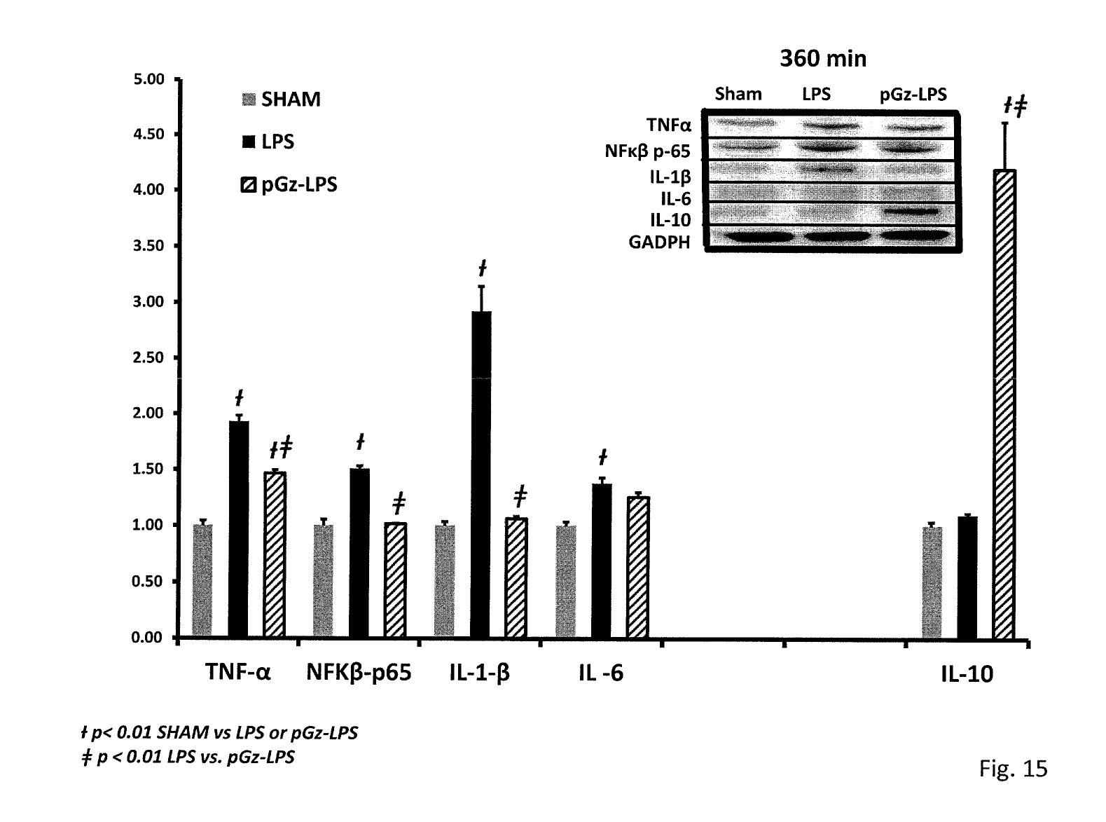

The graphs of FIGS. 14 and 15 depict in mice heart muscle Western Blot values in Optical Units (O.U.) referenced to protein/GADPH/SHAM at 90 and 360 minutes after initiation of investigation.

Interpretation of Cytokine and Inflammatory Factors changed by LPS and pGz.
Note that the Y axis scale of the 90 minutes graph is 70% of the 360-minute graph.
IL-1 beta, the most important inflammatory cytokine was lower at 360 than 90 minutes with pGz-LPS along with higher values of IL-10, an anti-inflammatory cytokine.
Inflammatory factors, TNF-alpha and NF-kB had similar values at 90 and 360 minutes.
Conclusion: Early initiation of pGz in the LPS sepsis model appears to prevent or minimize rise in inflammatory cytokines and inflammatory factors.

The effects of pGz pre- and postconditioning on TIE2 expression/phosphorylation and microvascular permeability are illustrated in the graphs of FIGS. 16 and 17.

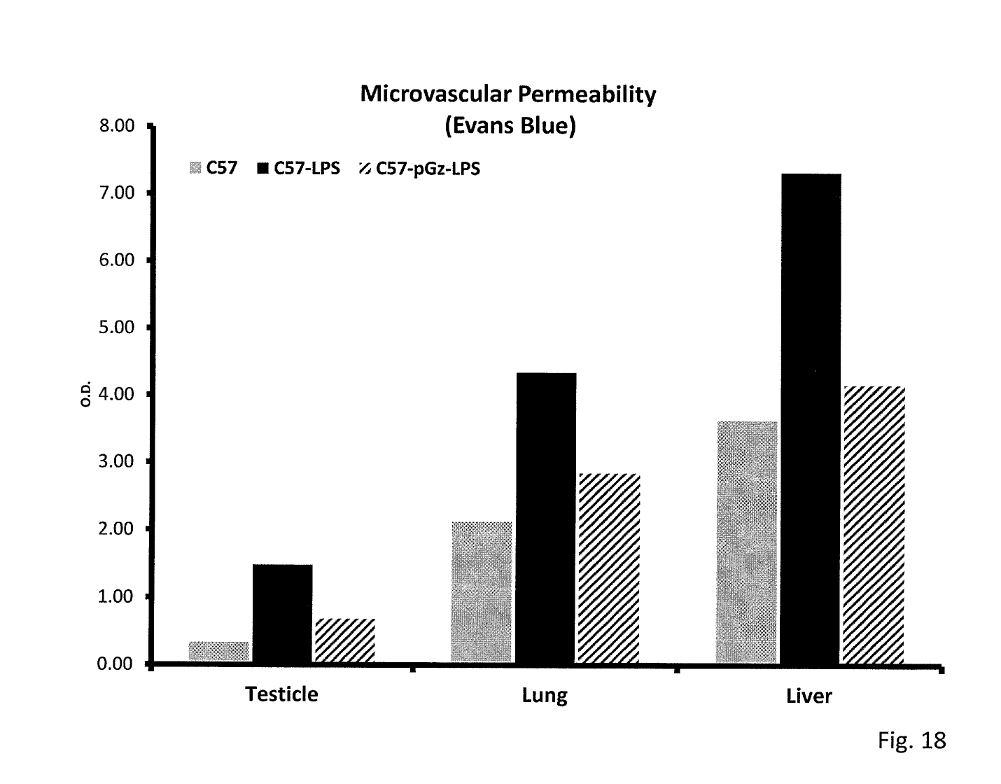

Interpretation of TIE2 in LPS Mice when Preconditioned
TIE2 is a mediator upregulated by endothelial derived nitric oxide that promotes tightening of endothelial intracellular junctions.
No changes of TIE2 content occurred at 90 and 360 minutes.
Phosphorylated TIE2 (pTIE2), the active form of TIE2 was lower during LPS and pGz-LPS at 360 compared to 90 minutes.
Conclusion: The lower values of pTIE2 at 360 minutes implies the possibility that supply might becoming exhausted and greater activity is needed.
FIG. 18 illustrates the effects of preconditioning on microvascular permeability with pGz at 360 minutes of in vivo injection of Evans Blue dye in mice to estimate fluid leakage from destruction of the endothelial barrier through post-mortem analysis
FIG. 18 depicts results of in vivo injection of Evans Blue dye in mice to estimate fluid leakage from destruction of the endothelial barrier through post-mortem analysis.
pGz diminished leakage from the endothelial barrier in the organs studied, e.g., testicle, lung and liver.

### CONCLUSION

Through its beneficial effects on release of several mediators including endothelial derived nitric oxide, prostacyclin, TIE2 among others, pGz provides protection to the endothelial barrier in the LPS mice model.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

We claim:

1. A method of treating a patient for septic shock or preventing sepsis in the patient by using a motorized machine, said machine having:
a housing;
an axis-defining mechanism coupled to the housing, the axis-defining mechanism configured to define a rocking axis;
at least one pedal positioned to receive a foot of the user and mounted on the rocking axis for rocking movement of the at least one pedal; a motor arranged within the housing, the motor configured to generate rotational motion to an output shaft of the motor;
a pedal rocking mechanism coupled to the output shaft and driven by the motor, the pedal rocking mechanism being configured to translate the rotational motion generated by the motor to reciprocating rocking motion of the at least one pedal about the rocking axis; and
at least one bumper, height-adjustably coupled to the housing, located spaced apart from a bottom portion of the at least one pedal, the reciprocating rocking motion of the at least one pedal provided by the pedal rocking mechanism providing a positive application of force for moving the bottom portion of the pedal towards and away from the at least one bumper, wherein the motor, the pedal rocking mechanism, the at least one pedal and the at least one bumper are configured so as to cooperate to apply, by the pedal rocking mechanism, the positive application of force to the at least one pedal so as to, during operation of the motor, cause the bottom portion of the at least one pedal to tap against the at least one bumper so as to provide pulsatile acceleration to the bottom of the user's foot, said pulsatile acceleration having a force sufficient to increase pulsatile shear stress to the endothelium, of sufficient magnitude to cause the release of beneficial mediators; the method comprising the steps of:
a) placing the patient in one of a seated and supine posture with the patient's foot on the at least one pedal;
b) securing the patient's foot to the at least one pedal; and
c) applying power to the device to cause the at least one pedal to move, at a therapeutically-effective speed, for a therapeutically-effective amount of time, towards and away from said bumper to cause the pedal to contact said bumper.

2. The method of claim 1, wherein step a) comprises placing the motorized machine at one of on a floor about 12″ from a chair containing the patient, and at a foot of a bed containing the patient.

3. The method of claim 1 wherein step b) comprises the use of straps having hook and loop fasteners.

4. The method of claim 1, wherein said motorized machine comprises two pedals and wherein step b) comprises each one of the patient's feet to a respective pedal.

5. The method of claim 1 wherein said motorized machine has a speed adjustment control.

6. The method of claim 5, wherein the speed adjustment control comprises a Bluetooth receiver.

7. The method of claim 1, wherein in the case of sepsis prevention, the method is performed twice daily for the therapeutically-effective amount of time.

8. The method of claim 7, wherein in the case of potential sepsis exposure, the method is performed twice daily, each time for the therapeutically-effective amount of time, the therapeutically-effective amount of time being at least 30 minutes, said twice daily performance of the method beginning at least three days prior to the potential exposure to sepsis.

9. The method of claim 1, wherein in the case of sepsis treatment, the method is performed upon detection of one of an unexpected fever, elevated heart rate and respiratory rate of the patient.

10. The method of claim 9, wherein the method is performed at least 30 minutes, four times a day, or continuously, or continuously, depending upon clinical response of the patient.

11. The method of claim 10, wherein the method is performed according to trends in Augmentation Index of the derived central aortic waveform; if values are less than 1%, then continuous operation of the device must be utilized.
